(19) United States
(12) Patent Application Publication
(10) Pub. No.: US 2007/0255294 A1
(43) Pub. Date: Nov. 1, 2007

(54) Medical Device with Enhanced Effect of Cell Regeneration and the Method Thereof

(76) Inventor: Wan-Seok Han, Seoul (KR)

Correspondence Address: BIRCH STEWART KOLASCH & BIRCH, PO BOX 747, FALLS CHURCH, VA, 22040-0747, US

(21) Appl. No.: 11/597,056

(22) PCT Filed: Dec. 6, 2005
(86) PCT No.: PCT/KR05/04164

§ 371 Date: Nov. 20, 2006

(30) Foreign Application Priority Data
Dec. 6, 2004 (KR) 10-2004-0101867
Dec. 6, 2004 (KR) 10-2004-0101838

(51) Int. Cl. A61B 17/06

(52) U.S. Cl. 606/139; 606/228

(57) ABSTRACT

The present invention relates to a medical device with a cell-regenerating effect and its manufacturing method. In detail, this device is manufactured by coating polydioxanone monofilament with collagen. The medical device enhances cell regeneration as it stimulates the closure of the incised area, and it shows a hemostatic effect and improves skin elasticity. The device is composed of a suture with biocompatibility, appropriate tensile strength and excellent sliding feature. In the device, one side of the needle is equipped with a handle while the other side is equipped with a sharp upper part that is inserted into the body, and the central space, including the internal part of the needle and the central part of the handle, are penetrated by the suture.

### TECHNICAL FIELD

The present invention relates to a medical device with a cell-regenerating effect and its manufacturing method. In detail, this device is manufactured by coating polydioxanone monofilament with collagen. The medical device enhances cell regeneration as it stimulates the closure of the incised area, and it shows a hemostatic effect and improves skin elasticity. The device is composed of a suture with biocompatibility, appropriate tensile strength and excellent sliding feature, and a medical device whose needle is equipped with a handle on one side and a sharp front end on the other, which is inserted into the body, and there is an inner space for the suture, which penetrates through the needle and the handle in the middle.

### BACKGROUND ART

Various sutures are currently being used in surgical operations to stitch organs such as blood vessels, internal organs and skins. These sutures can be classified into synthetic sutures and natural sutures, depending on the raw material. Depending on whether it can be absorbed into the body, sutures can also be classified into absorbable sutures that naturally decompose and are absorbed after a certain period of time after the cut had been stitched, and non-absorbable sutures that do not disappear naturally, thus necessitating a consequent operation to remove the suture. In addition, they can be divided into monofilaments and multifilaments, according to shape.

Absorbable sutures do not leave any alien substance in the sutured incised wound after they are absorbed, thereby facilitating recovery to the normal state without the necessity of a consequent operation. Thus, they are the most commonly used in operations these days. These sutures require biocompatibility and appropriate tensile strength. In addition, in order to minimize the friction that occurs when passing through the organs or fixing them after making a knot, an excellent surface sliding feature is also required for these sutures.

Previous patents on sutures include Korean Patent Application No. 2003-0041373 for high-tensile PGA/PLA copolymer sutures, and Japanese Public Patent No. 11-9678 for peptide-containing sutures for human organs. U.S. Pat. No. 3,736,646, No. 3,875,937 and No. 4,027,676 were for absorbable multifilament sutures with enhanced knotting feature.

However, various researches on the development of technologies for the manufacture of advanced sutures with biocompatibility, tensile strength, surface sliding feature, as well as the ability to enhance the closure of the wounded area, have been conducted. After exerting efforts to develop the most effective suture with all the aforementioned advantages, we, the inventors, have come up with the idea of combining polydioxanone and collagen.

Polydioxanone is used as a material for monofilament sutures. Since it is hydrolyzed into CP₂ and H₂O inside the human body and excreted afterwards, it is non-toxic to human bodies. Existing absorbable sutures are made from polydioxanone alone. However, they do not possess sufficient stitching strength when the fibers are thinner, and they do not have adequate tensile strength as well. In addition, enhanced closure of the wounded area and improved skin elasticity cannot be expected from these conventional sutures.

On the other hand, collagen is a kind of fibrous protein that exists in large amounts in the skin, bones, cartilages, arterial walls, teeth and muscles. The naturally derived soluble collagen is dissolved and absorbed inside the human body. Collagen is also used in the prevention of the effects of aging and in smoothing out wrinkles since it contributes in improving the appearance of the skin cells when injected into the body.

At present, no technology has yet been reported regarding the manufacture of sutures with superb functions through the effective combination of polydioxanone and collagen.

In general, medical needles are used for closing the incised skin or in plastic surgeries such as wrinkle-removal and double eyelid surgeries. These medical needles are manufactured in various forms.

Normally, one side of the needle is equipped with a sharp upper part, whereas the other side has a needle eye which is threaded with the suture. Most needle eyes are blunt so that the operator can hold them.

Previous patents regarding medical needles include Patent Register No. 0319209 for a dual-blade surgical needle whose plural number of needle eyes are in the central part of the needle through which the suture penetrates, and Patent Register No. 0344757 for a stitching device for laparoscopy in which the suture is easily hung on the load when the fiber is twisted with the load turning around inside the laparoscopy.

Medical needles should be designed in a way that considers the convenience of the operator, alleviates the pain of the patient, and stimulates cell regeneration. However, the existing technologies presented are deemed insufficient in satisfying these functions and difficulties in commercializing the technologies are experienced since the designs are too complicated.

Due to these reasons, we have strived to develop more advanced medical devices that guarantee easier use for operators, higher success rates in operations, simpler composition and more economic features. As a result, we were able to develop a medical device in which one side of the needle is equipped with a handle while the other side is equipped with a sharp upper part, which is inserted into the body, and the central space, including the internal part of the needle and the central part of the handle, is penetrated by the suture.

### DISCLOSURE OF THE INVENTION

### Technical Problems

The invention aims to provide sutures with biocompatibility, tensile strength and smooth sliding feature, as well as the effects of regenerating cells by facilitating hemostatic effects and skin elasticity, so that the stitched area recovers rapidly. This invention also aims to present the method of manufacture for the abovementioned cell-regenerating sutures.

We have discovered that we could manufacture a suture by dissolving and coating collagen onto polydioxanone. Thus, it stimulates the closure of the stitched area, enhances the hemostatic effect and skin elasticity, and enjoys high biocompatibility, tensile strength and smooth sliding feature.

In addition, the purpose of this invention could be accomplished by developing a medical device in which one side of the needle is equipped with a handle while the other side is equipped with a sharp upper part that is inserted into the body, and the central space, including the internal part of the needle and the central part of the handle, is penetrated by the suture.

### Technical Solutions

The invention relates to a medical device with cell-regenerating effects and its manufacturing method.

The aforementioned cell-regenerating medical device includes cell-regenerating sutures and a medical device that is convenient to use in surgical operations and plastic surgeries such as wrinkle-removal and double eyelid surgeries.

The aforementioned suture in this invention is manufactured in a manner that coats polydioxanone monofilament with collagen, and it exhibits excellent effects in stimulating the recovery of the wounded area and enhancing hemostasis and skin elasticity. Some of its other advantages also include excellent biocompatibility, tensile strength and smooth sliding feature.

The aforementioned medical device has a unique and efficient structure, in which one side of the needle is equipped with a handle while the other side is equipped with a sharp upper part that is inserted into the body, and the central space, including the internal part of the needle and the central part of the handle, is penetrated by the suture.

The suture in this invention can be made by coating the suture (polydioxanone monofilament) with collagen.

Polydioxanone has been used as a single material for existing sutures. Therefore, conventional sutures are available for coating with collagen. However, it is also possible to manufacture polydioxanone monofilament directly by melting polydioxanone and spinning the suture through a nozzle.

A 100%-pure collagen in the market can be used for this device. Either natural marine collagen or mammal-derived collagen may be used.

To manufacture the suture for surgical operations in this invention, first, the collagen coating solution is manufactured. Collagen is melted in 50˜60° C. water, which means that collagen accounts for 40˜70% of the solution in weight. If the amount of collagen does not reach the abovementioned range, the amount of collagen adhering to the polydioxanone monofilament is also small, resulting in the failure to enhance tensile strength, stimulate the fast closure of wounds, and improve skin elasticity. If the amount of collagen is greater than the abovementioned range, too much collagen adheres to the polydioxanone monofilament, which adversely affects the sliding feature of the suture.

Prepare the polydioxanone monofilament and bind it on a cylinder-type bobbin or drum. Attach it to a stirrer, and soak it in the collagen coating solution, and then stir it for a certain period of time.

When using a cylinder-type bobbin or drum with a hole or a stick-shaped drum, the coating effect can be enhanced since the coating solution can reach all of the sutures that wind up inside.

The polydioxanone monofilament coated with the collagen coating solution can be dried either in room temperature or by using a heater when deemed necessary.

With the manufacturing procedure described above, collagen-coated polydioxanone sutures can be manufactured with the effects of stimulating the closure of the stitched area and enhancing hemostasis and skin elasticity, thus ultimately facilitating cell regeneration. Their specific features also include excellent biocompatibility, tensile strength and sliding feature, which make them particularly suitable for surgical operations.

As for the operational medical device in this invention, the upper handle is in the form of a semicircle or equilateral triangle. The abovementioned suture is manufactured in the following method: collagen is melted in pure water to attain a 40˜70% (in weight) collagen-containing coating solution; the polydioxanone monofilament is soaked in the collagen-coating solution and stirred and dried. The abovementioned needle has an external diameter of 0.15˜0.18 mm and an internal diameter of 0.06˜0.08 mm.

The medical device in this invention is manufactured in a manner that guarantees the most appropriate and convenient form for an operator to perform a plastic surgery, such as wrinkle removal and double eyelid surgery, as well as other surgical operations with stitches. In addition, since it minimizes the pain of the organ undergoing the operation and stimulates cell regeneration, the device significantly contributes to higher success rates in surgical operations.

### BRIEF DESCRIPTION OF THE DRAWINGS

FIG. 1 is a perspective drawing of the semicircular section of the handle of the medical device.

FIG. 2 is a perspective drawing of the equilateral triangular section of the handle of the medical device, and the enlarged drawings of the section in each part.

FIG. 3 is a perspective drawing of the package that covers the medical device.

FIG. 4 describes how the operator uses the medical device in this invention.

### BEST MODE

In the following descriptions, enforcement and experimental examples of the specific compositions and functions of the invention are explained in greater detail. However, the descriptions below aim only to present examples of the invention, and it does not mean that the rights and criteria of the invention are only limited to the following examples.

### EXAMPLES 1˜6

Bovine derived collagen is melted in 50° C. pure water with 30, 40, 50, 60, 70, and 80% collagen in weight in order to manufacture the collagen coating solutions. The bobbins wound up with polydioxanone monofilament are soaked in the collagen coating solutions for 30 minutes, stirred and dried at 22° C. to attain collagen-coated polydioxanone sutures.

Experiment 1: Measuring the Sliding Feature and Friction Coefficient of the Sutures

The knot-sliding features and friction coefficients were measured on sutures manufactured through Examples 1˜6.

To measure the sliding feature of the knot, a knot was made in a 1×1 type among the knotting types suggested by Tera for surgical operations. The knot was made around a cylinder-type stick and then slid. This is a very simple and convenient method to measure sliding after knotting. In order to understand knot-sliding in damp conditions, it was measured after the suture was soaked in 25° C. water for one minute.

The friction coefficient was measured in quantity with an F-meter (Model: R-1182, manufactured by Rothschild (Switzerland)).

The experimental results are described in Table 1. Taking into account the results of the sliding effects, it is deemed desirable to manufacture sutures by using a collagen coating solution with 70% or lower in weight.

TABLE 1
Sliding feature and friction coefficient of the sutures

| | Knot sliding | Friction coefficient |
|---|---|---|
| Example 1 | ++ | 0.17 |
| Example 2 | ++ | 0.18 |
| Example 3 | ++ | 0.22 |
| Example 4 | ++ | 0.25 |
| Example 5 | + | 0.27 |
| Example 6 | − | 0.35 |

Note
++: very good,
+: good,
−: mediocre,
−−: bad

Experiment 2: Measuring the Tensile Strength After Suture Treatment

10-week-old male rats were prepared with their backs shaved. The center of the back was cut by 2 cm in a vertical direction. By using the sutures attained from Examples 1˜6, the incised area was closed with five stitches with the same intervals. After two weeks, the rats were sacrificed and the skins that underwent the operation were extracted, with a width of 1 cm to the left and the right from the center of the incised area. Then, the tensile strength was measured on the skin using a Rheometer. Five rats were measured for each suture, and the average figure is shown in Table 2. The more collagen in the solution, the higher the tensile strength attained. This proves the excellent function of the suture in this invention in facilitating the conjugation of the stitched area.

TABLE 2
Tensile strength of sutures (g/cm)

| | Example | Example | Example | Example | Example | Example |
|---|---|---|---|---|---|---|
| | 1 | 2 | 3 | 4 | 5 | 6 |
| Tensile | 191 | 214 | 225 | 232 | 251 | 258 |
| strength (g/cm) | | | | | | |

Experiment 3: Measuring the Effects in Skin Elasticity and Wrinkle Removal After Suture Treatment

Elasticity was measured on the back of 10-week-old male rats using a Cutometer (SEM474, Courage+Khazaka electronic GmbH, Germany). Afterwards, the backs of the rats were shaved and cut by 2 cm from the center in a vertical direction with a surgical knife. The incised areas were closed with five stitches with the same interval using the abovementioned sutures in Examples 1˜6. After 2 weeks, elasticity was measured again on the backs using a Cutometer (SEM474, Courage+Khazaka electronic GmbH, Germany). Five rats were measured for each suture. The results of the measured elasticity were compared before and after the suture was used, and then the elasticity improvements were calculated in percentage(%) and presented in Table 3. The results indicate that the suture in this invention shows an outstanding ability in improving skin elasticity, and that it is desirable to manufacture sutures with collagen weighing more than 40% in the collagen coating solution to ensure such ability.

TABLE 3
Improved skin elasticity by sutures (%)

| | Example | Example | Example | Example | Example | Example |
|---|---|---|---|---|---|---|
| | 1 | 2 | 3 | 4 | 5 | 6 |
| Elasticity | 4 | 12 | 17 | 21 | 23 | 24 |
| improvement | | | | | | |
| (%) | | | | | | |

Experiment 4: Measuring the Hemostatic Effect After Suture Treatment

Human blood was placed in test tubes and treated with heparin, and then the sutures obtained from Examples 1˜6 were put in each of the test tubes to examine blood clotting using the naked eye. A control group with no suture was compared with the test tubes. The results showed that there was no change in the control group without a suture, whereas blood clotting occurred in the test tubes with sutures.

The attached drawings can explain the operational medical device in this invention in greater detail.

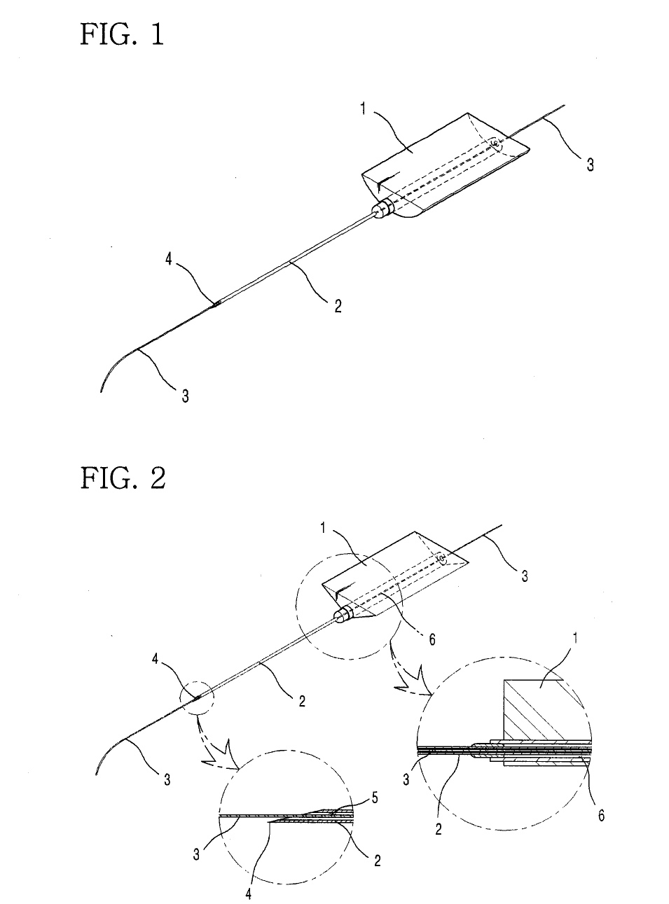

FIG. 1 is a perspective drawing of the semicircular section of the handle of the medical device. FIG. 2 is the enlarged drawings of each part of the medical device.

The medical device in this invention is composed of the handle (1), the needle (2), which is linked to the handle and inserted into the human body, and the suture (3), which penetrates the internal space of the needle and the center of the handle.

The upper part of the needle (2) is composed of a sharp, tapered area to be inserted into the human body, whereas the inside forms a space (5) so that the suture (3) can go through it.

The section of the handle (1) is either semicircular or equilateral triangular. The central part has a space (6) linked to another space of the needle (5) so that the suture (3) can go through it.

FIG. 1 shows the semicircular section of the handle (1), whereas FIG. 2 shows the equilateral triangular section.

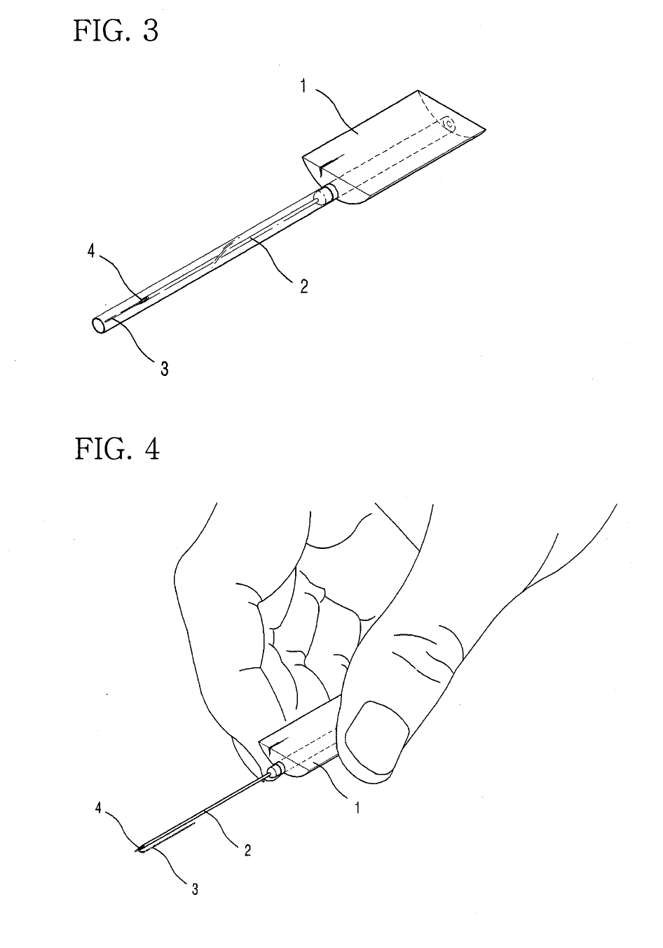

FIG. 3 is a perspective drawing of the package covering the medical device. As described here, the medical device in this invention is packaged with a cover made of a plastic tube, so that the sharp area of the needle is safely stored.

FIG. 4 describes how the operator uses the medical device in this invention.

The method for inserting the suture into the human body using the medical device in this invention is described as follows:

As shown in FIG. 4, grip the handle (1), bend the upper part of the suture (2), and insert the insertion part (4) into the target area of the human body. Have the needle go through the target area so that the suture (3) is inserted into the human body with the needle (2). Then, take out the needle to the original position and cut the left out suture with a scissor. This way, the suture is inserted in the target area to stimulate fast closure and cell regeneration.

The handle (1) is made from rubber, plastic or metal. The ideal diameter of the section of either the semicircle or the base of the equilateral triangle is 2.5˜3.5 cm.

The section of the handle is either semicircular or equilateral triangular, and its diameter should fall within the abovementioned range. As seen in FIG. 4, it is easy for an operator to hold the handle with one hand and accurately place the device onto the target area. It also makes the operator's hand less fatigued during operations.

The needle (2) is made from metal, and it is desirable to have an external diameter of 0.15˜0.18 mm and an internal diameter of 0.06˜0.08 mm.

Since the external and internal diameters of the needle are as minute as those described above, it minimizes the pain before and after the surgical operation. When the external and internal diameters are larger than the abovementioned range, it may be inappropriate for operations without anesthesia due to pain. On the other hand, when the diameters are smaller, it is not easy to insert the needle into the human body.

The suture (3) is obtained through the following methods:

Collagen is fused in pure water to make a 40˜70% collagen (in weight) coating solution. Polydioxanone monofilament is soaked in this collagen coating solution, stirred and dried to obtain the stitch fiber.

The suture is efficient for surgical operations due to its excellent biocompatibility, tensile strength and smooth sliding feature. In addition, it also facilitates cell regeneration since it stimulates the faster closure of the incised area, enhances hemostasis, and improves skin elasticity.

The medical device in this invention can enhance the convenience of the operator, alleviate the pain experienced by the patient, and facilitate cell regeneration to ultimately enhance the success of the operation.

### INDUSTRIAL APPLICABILITY

As described above, the innovative medical device manufactured according to this invention can significantly facilitate cell regeneration. In particular, the suture in this invention was proven to have excellent biocompatibility, tensile strength and smooth sliding, which are very useful for surgical operations. By stimulating the fast closure of the incised area, enhancing hemostasis and skin elasticity, the suture facilitates cell regeneration and brings about the advantages of health and beauty of the human body. The surgical device is designed to provide convenience to the operator during surgical stitching or plastic surgeries such as wrinkle removal and double eyelid surgeries. It is also designed to minimize the pain felt by patients and facilitate cell regeneration to significantly enhance the success of the operation. Thus, the medical device is deemed as a very useful device for the cosmetic and medical industries.

1. A manufacturing method of suture comprising the steps of:
melting collagen in pure water and obtaining a coating solution with 40˜70% collagen in weight; and
soaking polydioxanone monofilament in the collagen coating solution, stirring and drying it to obtain a suture.

2. A suture with cell-regenerating function manufactured in the method described in the claim 1.

3. A medical device, in which one side of the needle is equipped with a handle while the other side is equipped with a sharp upper part that is inserted into the body, and the central space, including the internal part of the needle and the central part of the handle, penetrated by a suture.

4. The medical device according to claim 3, wherein the shape of the section of the handle of the medical device is either in semicircular or equilateral triangular form.

5. The medical device according to claim 3, wherein the suture is the same to the suture for cell regeneration in claim 2.

6. The medical device according to claim 3, wherein the needle has an exterior diameter of 0.15˜0.18 mm and an interior diameter of 0.06˜0.08 mm.

7. A method of using the medical device in claim 3 by bending the upper part of the suture, inserting the upper part of the needle into the target area of the human body, having the needle go through the area, taking out the needle to the original position, and cutting the left out suture with a scissor.
